(19) United States
(12) Patent Application Publication
(10) Pub. No.: US 2017/0049368 A1
(43) Pub. Date: Feb. 23, 2017

(54) METHODS AND SYSTEMS FOR DETERMINING THE HEARING STATUS OF A PERSON

(71) Applicants: AUSTRALIAN HEARING SERVICES, Hornsby (AU); CMEE4 PRODUCTIONS PTY LTD, Hornsby (AU)

(72) Inventors: Harvey Dillon, Turramurra (AU); Caroyln Mee, Castlecrag (AU)

(21) Appl. No.: 14/989,221

(22) Filed: Jan. 6, 2016

(30) Foreign Application Priority Data
Aug. 18, 2015 (AU) 2015903334

(51) Int. Cl. A61B 5/12; A61B 5/00

(52) U.S. Cl. CPC A61B 5/123; A61B 5/7271

(57) ABSTRACT

Systems and methods for determining the hearing status of a person are described that involve administering more than one type of a hearing test in the context of a game or games.

### TECHNICAL FIELD

This patent application relates to systems and methods for behaviourally determining the presence and nature of hearing problems. It is particularly suitable for testing the hearing of children, but can be used for test persons of any age.

### BACKGROUND TO THE INVENTION

Universal new-born hearing screening has been spectacularly successful in detecting children born with hearing loss of moderate degree or greater. Despite this, more children receive their first hearing aids during the first three years of school than receive them during their first year of life. Some of these children have losses too mild to be detected at birth, some have progressive hearing loss, some have unilateral loss, and some have acquired hearing loss subsequent to birth through disease or trauma. Irrespective of the reason, these children typically are brought in for a hearing assessment because they are performing badly at school. All too often, their self-esteem and attitude to school have been adversely affected by the time a parent, teacher or other professional works out that hearing loss might be the cause of their problem and seeks an assessment. Screening the hearing of all children as they enter school is sufficiently expensive that in most places, it doesn't happen. Where it does occur, it is limited to detecting the presence of hearing loss, not the presence of auditory processing disorders that can occur despite normal hearing sensitivity.

### SUMMARY OF THE INVENTION

In a first aspect the present invention provides a method of determining the hearing status of a person that involves administering more than one type of a hearing test in the context of a game or games.

The results of the more than one hearing tests may be combined to calculate the presence or nature of the hearing problem.

One of the hearing tests may comprise a test of the ability to recognise speech sounds in quiet.

One of the hearing tests may comprise a test of the ability to recognise speech sounds in the presence of other sounds.

One of the hearing tests may comprise a test of the ability to detect tonal sounds in the presence of other sounds.

One of the hearing tests may comprise a test of the ability to detect tonal sounds in quiet.

Either the target sounds to be detected, or the one or more competing sounds, or both the target sounds and the one or more competing sound or sounds, may be processed so that the target sounds appear to come from a different direction than the competing sound or sounds.

The results of the more than one test may be combined in such a way that below-normal performance in one test combines with below-normal performance in one or more further tests to produce an overall test result that represents a greater deficit from normal than is indicated by the results of either test alone.

The scores for an individual may be expressed relative to those of a population known not to have any hearing problems.

The scores for an individual may be expressed in terms of population standard deviation units by dividing the difference between the score for a test person and the average score for test persons of a similar age by the standard deviation around the average for test persons of a similar age.

The results of the more than one tests may be combined in such a way that differences in performance between the different tests are used to predict the type of hearing problem that is most likely to be present.

The types of hearing problems that may be differentiated by comparing the results of the more than one test may include any one or more of sensorineural hearing loss, conductive hearing loss, auditory processing disorder, or language disorder.

The type of hearing problem most likely to cause the poor test results may be calculated by comparing, for the set of tests, the performance of the test person to the performance of groups of people with known types of hearing problems, and selecting the group to which the test person's results are most similar.

The person being tested may respond to the presentation of target sounds by selecting with a computer mouse, or other pointing device, or touching on a touch screen, visual images that represent the correct response to the target sound presented, or by making verbal responses, with such verbal response being recognised by voice recognition software within the test device.

The results for an individual test may be derived by comparing the results of the test person to the results of a reference person who is believed to have normal hearing ability and who undergoes a test using the same computing device and headphones.

The stimuli may be made equally difficult by analysing, across test participants, for each test item, the proportion of items correct as a function of its presentation level relative to the average presentation level for each participant, and then adjusting the level of each item so that the percentage correct for all the items have approximately the same dependence on presentation level relative to the average presentation level of all items.

The stimuli used to create the tests may be an integral part of the game or games played during the test.

The game may involve a story with characters or narrative or both, designed to maintain the interest, attention and concentration of the test person for the duration of the tests.

The results of the test or tests may be displayed to the test person immediately on completion of the test.

In a second aspect, the present invention provides a system for determining the hearing status of a person including: means for administering more than one type of a hearing test in the context of a game or games.

In a third aspect the present invention provides a computer program which includes instructions to cause a computing device to conduct a method according to the first aspect of the invention or to operate as a system according to the second aspect of the invention.

In a fourth aspect the present invention provides a computer readable medium that contains instructions according to the third aspect of the invention.

### BRIEF DESCRIPTION OF THE DRAWINGS

An embodiment of the present invention will now be described, by way of example only, with reference to the accompanying drawings, in which:

FIG. 1. shows an embodiment of a system according to the invention displaying a graphic from a game, in which some target stimuli are pictured on the screen, along with images of other objects that are not target stimuli;

FIG. 2. shows a flowchart detailing the main steps of an embodiment of a method according to the invention; and

FIG. 3. shows an adaptive track, showing the test signal to noise ratio versus the presentation number, from a test in which the difficulty of the task is adapted depending on whether the test person responded correctly or incorrectly or failed to respond, to the stimulus or stimuli presented previously in the test.

### DETAILED DESCRIPTION OF THE PREFERRED EMBODIMENT

This patent application describes a method and system for testing hearing without needing a trained person to administer the test. Sound stimuli are presented in the context of a game, to engage and retain the test person's attention during the test, even when the test person is a young child. The system has been implemented in both mobile technology and in a desk computer. As one goal is to ensure that children are able to understand their teacher in a typical classroom, the game is designed to be sensitive to not only hearing loss, but also other disorders that can impede understanding in the classroom. These include auditory processing disorders and language disorders. The game has also been designed to indicate the most likely cause of hearing difficulties, which necessitated including more than one type of hearing test within the game.

The operation of one embodiment of the invention will now be described. This embodiment has been implemented on both a personal computer and on a mobile tablet device. The embodiment combines a test of speech perception in quiet, a test of speech perception in noise, and a test of tone perception in noise, all in the context of a game in which the test person interacts with characters in the game by performing the tasks instructed to the test person by one or more characters in the game. Optionally, the suite of tests can also include a test of tonal sounds in quiet.

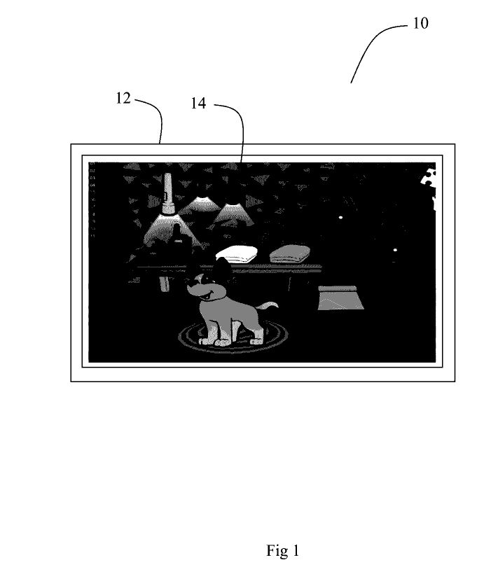

Referring to FIG. 1, a system 10 is shown in the form of a mobile tablet device 12 with a touch sensitive screen 14. Screen 14 displays an example of a graphic of a game in which some target stimuli are pictured on the screen including objects of various colours such as flashlights and cameras. During the game, the test subject is required to respond to sounds in the game by touching on the relevant object on the screen at the correct time.

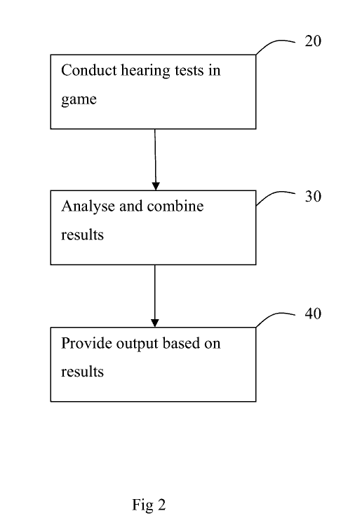

Referring to FIG. 2, the method conducted by system 10 begins at step 20 by conducting various hearing tests in the context of one or more games, the results of the tests are then analysed and combined at step 30 and at step 40 an output is provided based on the results.

### Speech in Quiet Game

Because the game may be played on an unknown tablet device using an unknown pair of headphones, no assumptions can be made about the absolute level of sound from the headphones. The speech in quiet game is aimed at determining the softest speech sound the test person can understand (expressed as the digital signal level sent to the sound card). This level is compared to the softest speech sound that a reference person with no known hearing loss can understand, but the accuracy of the overall game, as will be explained later, is not dependent on the reference person actually having good hearing. In the speech in quiet game, five differently coloured birds fly across the tablet screen. The test person is instructed to tap the bird whose colour matches the word (red, blue etc) that they hear. Following some practice trials during which an approximate threshold is established, the sound level is decreased (by 3 dB) following each correct tap and increased (by 3 dB) following each incorrect tap or failure to tap, until the speech reception threshold in quiet (SRTq) is found, calculated as the average of the levels presented after both correct and incorrect responses have previously occurred and at least 4 presentations have occurred. The difference between the test person and reference person's speech reception thresholds (ΔSRTq) provides one metric used in the final screening result, and the accuracy of this metric is of course dependent on the reference person having good hearing. The speech in quiet game is carried out separately for stimuli presented to each ear, via headphones. The presentation level for the competing sounds in each ear in the rest of the test is a predetermined number of decibels above the speech reception thresholds found for the test person in that ear. People with conductive loss or sensorineural loss are expected to have elevated speech reception thresholds in quiet.

An alternative method to construct the speech in quiet test is to limit application of the test to situations where it can be used with headphones of known (or readily calibrated) sensitivity, driven by computing devices with known (or readily calibrated) electrical output level.

### Speech-In-Noise Game

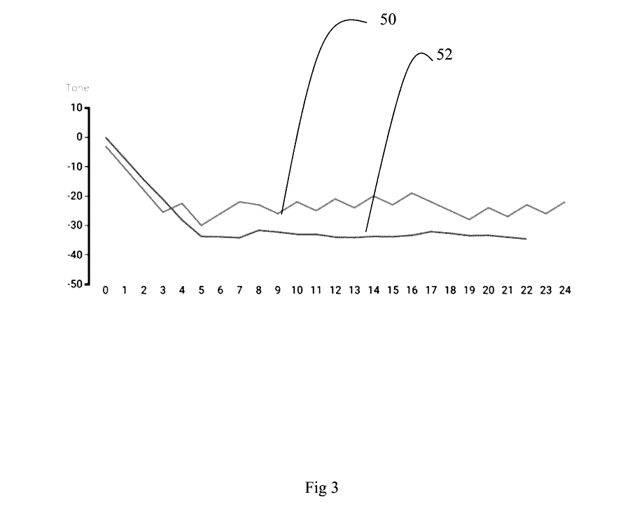

The speech-in-noise game requires the test person to tap on object on the screen (see FIG. 2.) as the names of the objects are called out. The objects form part of a story in which a park ranger sets out to locate a lost ranger, and in so doing requires the help of the person being tested. The background in which the objects are placed changes continuously as the story develops. To increase the sensitivity of the test to unilateral loss and spatial processing disorder, which is one form of auditory processing disorder, different competing sounds (each of which is ongoing speech from a single talker) are applied, seemingly coming from different directions. This is achieved, as is usual, by applying head-related transfer functions, corresponding to different directions, to each competing stimulus. The level of the target sound, and hence the signal-to-noise ratio (SNR) is adjusted adaptively, increasing by 4 dB after an incorrect response, increasing by 2 dB after an absent response, and decreasing by 2 dB after a correct response. When no response is made, the item is repeated twice before moving onto the next item, so the number of items presented is increased for more cautious test persons. If there are no failures to respond (just incorrect responses when uncertain), this combination of step sizes adapts to the 66% point on the test person's psychometric function. If there are no incorrect responses (just absent responses when uncertain), the test adapts to the 50% point on the psychometric function. Consequently, the target level is somewhere between 50 and 66% depending on the caution of the test person when items are not clearly heard. The speech reception threshold in noise (SRTn) is the average of the SNRs presented after completion of an adaptive practice phase during which a larger step is used following correct responses. Validity of the SRTn is evaluated by calculating the standard error of the presentation SNRs during the measurement phase, and deeming invalid any results for which the standard error exceeds 1.3 dB. This value is the upper limit found during extensive pilot testing for the great majority of children, for whom the adaptive process results in a well-behaved adaptive track (see FIG. 3.), showing the practice phase followed by small fluctuations in SNR around the child's threshold SRTn. Element 50 in FIG. 3 is the adaptive track for a particular child. The variations in that track are the “small fluctuations in SNR around the child's threshold SRTn” referred to in the preceding sentence. Element 52 is the average adaptive track for a large number of children.

Calculation of the standard error takes into account the non-independence of the presentation SNRs (as each SNR can vary from the previous one by only one step size). Reliable test scores are achieved by using at least 24 test items. To minimize the effect of guessing by the test person, the number of possible response options visible on the screen is always at least six. Test persons with sensorineural loss and some auditory processing disorders and language disorders are expected to perform outside normal limits on this test. Because the test result is expressed as the lowest level of the target items that enables the target items to be recognised, relative to the level of the competing sounds, the test result does not depend on knowing either the sensitivity of the headphones or whether the reference person who plays the speech in quiet game actually has normal hearing ability.

### Tone-In-Noise Game

The tone-in-noise game is similar to the speech-in-noise game, except that the target sound is a pure tone, frequency modulated between 1400 Hz and 1600 Hz (to simulate a bird call), presented to both ears, and the competing sounds (each one processed to appear to come from a different direction) are temporally modulated random noises, each of which has been band-stop filtered to have very little power between 1350 Hz and 1650 Hz. The two competing sounds (which in the story are “engine noises”) differ both in their spectral shape below 1000 Hz and above 2000 Hz (which does not affect their ability to mask the tonal sounds) and in the rate at which they are temporally modulated. They thus have a noticeably different character in each ear. The test person's task is to tap an on-screen button every time they hear a bird, for which they are rewarded with a “photo” of the bird. The step sizes used for the bird sound during the measurement phase are a 3 dB decrease when the sound is detected and a 4 dB increase when it is not detected. The test thus adapts to the 57% point on the tone detection psychometric function. The tone reception threshold in noise (TRTn) is the average of the SNRs presented after completion of an adaptive practice phase during which larger step sizes are used (7.5 dB decrease and 3 dB increase). Two validity checks are carried out. As with the speech-in-noise test, the standard error during the measurement phase is calculated and values greater than 2.0 dB are deemed invalid. In addition, tone-in-noise tests for which there are greater than a specified number of false button presses are deemed invalid. Reliable test scores are achieved by using at least 24 presentations of the tonal target stimuli. Test persons with sensorineural loss are expected to perform outside normal limits on this test, because sensorineural loss causes a widening of auditory filters, decreasing the test person's ability to focus on the narrow frequency region in which they quickly learn the target is located. People with sensorineural hearing loss also have reduced ability to detect sounds during the low-power portions of temporally modulated masking sounds. Because the test result is expressed as the lowest level of the target items that enables the target items to be detected, relative to the level of the competing sounds, the test result does not depend on knowing either the sensitivity of the headphones or whether the reference person who plays the speech in quiet game actually has normal hearing ability.

### Equivalence of Speech Items

To create items of equal difficulty, the results from 128 test persons using a preliminary version of the game were analysed as follows. For each presentation of a target stimulus, the presentation level was expressed relative to the final SRTₙ measured for that respondent, and the correctness of the response noted. These relative presentation levels were grouped into bins 2 dB wide, and for each bin, the proportion of times that each target sound was correct was calculated. These data were fitted with psychometric functions as given in Equation 1.

P=1/(1+e^{(a+b.L)} 1,

where P is the proportion correct, a and b are fitting constants, and L is the relative presentation level.

The same value of b (the best fitting value of which was 0.327) was used for all target sounds. The SNR at which individual curves passed through the 50% correct point varied from −5.2 dB up to 4.3 dB, which is a total range of 9.5 dB in variation of item difficulty. The relative presentation levels for 50% correct were then averaged across items, giving a mean value of −1.30 dB. To estimate the adjustment that should be made to each stimulus to equate their difficulty at the 50% correct point, the relative presentation level at 50% correct for each stimulus was then subtracted from the mean value. The levels of all stimuli were adjusted by these calculated amounts prior to further testing so that items in the speech-in-noise test have close to equal intelligibility. This process maximised the accuracy of the speech test by minimising the variation in SNR as the test progresses.

### Calculation of Hearing Deficit Scores for Each Test

Hearing deficit scores are first calculated separately for each of the three tests: speech-in-quiet, speech-in-noise, and tone-in-noise. For each test the same calculation methods are used to determine the deficit measured in normal-hearing population standard deviation units, often referred to as z scores. The steps are as follows.
1. The test measures (ΔSRTq, SRTn and TRTn respectively) for a population of people of different ages with normal hearing ability are regressed against the age of the test persons, using the exponential function shown in Equation 2.

Estimated test measure=c+d. e^{-f(age-g)} 2,
where c, d f and g are the fitting parameters.
2. The differences between the actual test results and the estimated test results (using equation 2 for each test measure) are calculated.
3. The standard deviation of the differences calculated in step 2 are calculated.
4. The individual z-score for each test person is equal to the difference between the person's actual score and the estimated score for a person of that age with normal hearing ability, with this different divided by the standard deviation found in step 3.

These individual test results are individually meaningful to clinicians, but can be combined, as described in the following text, to produce a metric that is both more meaningful to lay people and also more accurate because it combines the results of several tests.

### Calculation of Overall Hearing Ability

The overall hearing ability can be calculated from the hearing deficit scores for each test by combining these scores in many different ways. These methods include, but are not limited to:
1. The overall hearing deficit can be estimated as the individual test score that shows the greatest deficit, as shown in equation 3.

Overall hearing deficit=Min(ΔSRTq_{z-score},SRTn_{z-score},TRTn_{z-score}) 3.
2. The overall hearing deficit can be estimated as the sum of the individual test scores, as shown in equation 4.

Overall hearing deficit=ΔSRTq_{z-score}+SRTn_{z-score}+TRTn_{z-score} 4.
Optionally, the individual test z-scores used in equation 4 can be limited to a maximum value of zero, prior to application of equation 4, so that tests in which the test person has greater than average ability do not offset the deficits measured in tests for which the test person has poorer than average ability.
3. A calculation method intermediate to the two preceding methods, in which all tests potentially contribute to the overall deficit, but in which the test with the poorest score contributes more strongly than the other tests, is to square, or raise to some other power greater than one, the individual test scores before they are combined, such as the equation shown in equation 5.

Overall hearing deficit=√(ΔSRTq_{z-score}²+SRTn_{z-score}²TRTn_{z-score}²) 5,
where the individual test z-scores used in equation 5 are limited to a maximum value of zero, prior to application of equation 5, so that tests in which the test person has greater than average ability do not offset the deficits indicated in tests for which the test person has poorer than average ability. Alternative versions of equation 5 can be composed in which the individual test scores are not limited to a maximum value of zero.
4. The overall hearing deficit score, such as any one of those calculated in the preceding three steps, can be displayed as the test result, or can be used to derive a more meaningful test result, such as by using one of the following methods.
   a) The test-person's overall hearing deficit score can be compared to those of a group of people with normal hearing ability, and the test person's score can be expressed as a percentile score representing that person's position within the distribution of scores of those with normal hearing ability.
   b) The mean and standard deviation of the overall hearing deficit scores for a group of people with normal hearing ability can be estimated. A z-score can then be calculated for a test person by subtracting the test person's overall hearing deficit score from the normal hearing population mean overall hearing deficit score and dividing the difference by the standard deviation of the scores for the normal hearing population.
   c) The z-score calculated in (b) can be expressed as a standard score, such as by multiplying the z-score by 15 and adding the result of this to 100.
   d) A linear regression can be formed between overall hearing deficit scores and hearing loss, averaged across selected frequencies, for people with known degrees of hearing loss. This regression can then be used to estimate the degree of hearing loss, in units of decibel hearing level, that would typically result in the overall hearing deficit score achieved by that test person. The result can then be expressed as the test person's equivalent hearing threshold.

### Estimation of Likely Cause of Hearing Difficulty

Because the different types of tests will be differently affected by different types of hearing problems, a comparison of test results across tests can be used to infer the most likely cause of a hearing deficit. This comparison relies on the following principles.
1. Sensorineural hearing loss is likely to cause abnormal performance for speech in quiet, speech in noise, tone in quiet and tone in noise.
2. Conductive hearing loss, including conductive hearing loss caused by middle ear infections, is likely to cause abnormal performance for speech in quiet, and tone in quiet, but not for speech in noise or tone in noise if the presentation level of the stimuli is increased to compensate for the conductive hearing loss.
3. Auditory processing disorders are likely to cause abnormal performance for speech in noise, and may cause abnormal performance for tone in noise depending on the nature of the auditory processing disorder.
4. A language disorder, or having poor proficiency in the language in which the test is constructed, such as might occur for those for whom that language is not their native language, is likely to cause abnormal performance for speech in noise, but not for tone in quiet or tone in noise.

One method for inferring the most likely cause of the hearing problem is to compare the results of individual tests with the results expected to occur on theoretical grounds, such as those described in the above four points. An inference that the hearing problem is of a particular type can be drawn whenever the results on each test fall below the score for that test for people with no hearing problems by some predetermined amount, with the predetermined amount chosen based on the expected effect of each type of hearing problem on each test.

An alternative method of inferring the most likely cause of the hearing problem is to compare the test person's scores to those of people for whom the cause of their hearing problems is known. For each person with a known type of hearing problem, their performance on n different tests can be characterised by expressing the n test z-scores as co-ordinates in n-dimensional space. The co-ordinates of a group of such people with the same type of hearing problem can be summarised as the mean co-ordinates for the group (a vector of dimension n), along with the standard deviations of the scores for each test (a second vector of dimension n). Each type of hearing problem will therefore give rise to a cluster of points (one for each test person) in n-dimensional space. Each cluster is characterised by its mean co-ordinates and the standard deviation around the mean in each dimension. A test person is most likely to have a hearing problem of the type whose cluster is closest to the results of the test person. The Euclidean distance from the test-person's results are calculated by subtracting, in each dimension, the test person's score from the cluster mean, and then dividing that difference by the cluster standard deviation in that direction. These ratios are combined by squaring each, adding the squares, and taking the square root of the sum. This value can be thought of as the Euclidean distance, normalised by the group standard deviations in each dimension, from the test person's results to each hearing problem group. The test person is then inferred to have a hearing problem of the type for which the Euclidean distance is smallest. Optionally, one of the groups for which a cluster is located in the n-dimensional space, is a group with completely normal hearing.

An alternative method that is intermediate to these methods is to determine rules that describe the minimum performance deficit on each test expected for people with each type of hearing problem, with the numerical cut-off values used in these rules determined on the basis of groups of people known to have each type of problem, optionally including a group of people known to not have any of the problems.

Interaction of Tests with Game

The tests described previously can be integrated with a game designed to maintain the interest and attention of the test person for the duration of the tests. With this method, the test can be made suitable for testing the hearing ability of children without requiring the tests to be administered by, or supervised by, a clinician skilled in the measurement of hearing ability. Optionally, the game can be based on a narrative, such that the target sounds and objects form part of the story described by the narrative. Variations to the language and themes used in the game can be made so that different versions of the game are optimised to attract and maintain the interest of test persons of different ages, from young children through to very elderly people.

To further maintain attention, the rules that control adaptation of the stimuli during the tests can be chosen such that stimuli are correctly perceived a high proportion of the time. Methods for achieving this include:
1. Adopting an asymmetrical step size, such that the level of the stimulus is increased by a greater amount following an incorrect or missing response than it is decreased following a correct response;
2. Reducing the level of the stimulus only after j items in a row elicit correct responses, and increasing the level of the stimulus only after k items in a row elicit incorrect responses or fail to elicit a response, where j is a larger number than k.
   Those skilled in the art will realise that instead of varying the level of the target stimulus, the level of the competing sound or sounds can instead be varied, in which case the rules governing level change in the preceding two points are changed to the following:
3. Adopting an asymmetrical step size, such that the level of the competing sound or sounds is decreased by a greater amount following an incorrect or missing response than it is increased following correct response;
4. Increasing the level of the competing sound or sounds only after j items in a row elicit correct responses, and decreasing the level of the competing sound or sounds only after k items in a row elicit incorrect responses or fail to elicit a response, where j is a larger number than k.

To further maintain interest and encourage appropriate responses to the stimuli, a visual reward system is built into the tone-in-noise game to reinforce the participant correctly identifying the tonal target stimuli.

Because of the effectiveness of the game in maintaining the interest and attention of test persons, the game is able to be played reliably by children down to the age of 4 years. It is thus able to be used to detect auditory processing disorders in children younger than can be detected using any other available tests.

Because the level of the target stimuli in each test adapts to the abilities of the test person, people with hearing ability across a broad range will be able to complete the game, and therefore will not be disqualified from participating in the entire narrative journey/game experience.

Where methods and apparatus of the present invention may be implemented by software applications, or partly implemented by software, then they may take the form of program code stored or available from computer readable media, such as CD-ROMS or any other machine readable media, the program code comprising instructions which, when loaded onto a machine such as a computer, the machine then becomes an apparatus for carrying out the invention. The computer readable media may include transmission media, such as cabling, fibre optics or any other form of transmission media.

It will also be appreciated that, where methods and apparatus of the present invention are implemented by computing systems, or partly implemented by computing systems, then any appropriate computing system architecture may be utilised. This will include stand-alone computers, networked computers, and dedicated computing devices. Where the terms “computing system” and “computing device” are used, then these terms are intended to cover any appropriate arrangement of computer hardware for implementing the function described.

Any reference to prior art contained herein is not to be taken as an admission that the information is common general knowledge, unless otherwise indicated.

Finally, it is to be appreciated that various alterations or additions may be made to the parts previously described without departing from the spirit or ambit of the present invention.

1. A method of determining the hearing status of a person including the steps of:
administering more than one type of a hearing test in the context of a game or games.

2. A method according to claim 1 further including the step of combining the results of the more than one hearing tests to calculate the presence or nature of the hearing problem.

3. A method according to claim 1 wherein one of the hearing tests comprises a test of the ability to recognize speech sounds in quiet.

4. A method according to claim 1 wherein one of the hearing tests comprises a test of the ability to speech sounds in the presence of other sounds.

5. A method according to claim 1 wherein one of the hearing tests comprises a test of the ability to detect tonal sounds in the presence of other sounds.

6. A method according to claim 1 wherein one of the hearing tests comprises a test of the ability to detect tonal sounds in quiet.

7. A method according to claim 4 where either the target sounds to be detected or the one or more competing sounds, or both the target sounds and the one or more competing sound or sounds, are processed so that the target sounds appear to come from a different direction than the competing sound or sounds.

8. A method according to claim 2 wherein the results of the more than one test are combined in such a way that below-normal performance in one test combines with below-normal performance in one or more further tests to produce an overall test result that represents a greater deficit from normal than is indicated by the results of either test alone.

9. A method according to claim 1 in which the results for an individual are expressed relative to those of a population known not to have any hearing problems.

10. A method according to claim 9 in which the results for an individual are expressed in terms of population standard deviation units by dividing the difference between the score for a test person and the average score for test persons of a similar age by the standard deviation around the average for test persons of a similar age.

11. A method according to claim 2 wherein the results of the more than one tests are combined in such a way that differences in performance between the different tests are used to predict the type of hearing problem that is most likely to be present.

12. A method according to claim 11 wherein the types of hearing problems that are differentiated by comparing the results of the more than one test include any one or more of sensorineural hearing loss, conductive hearing loss, auditory processing disorder, or language disorder.

13. A method according to claim 11 in which the type of hearing problem most likely to cause the poor test results is calculated by comparing, for the set of tests, the performance of the test person to the performance of groups of people with known types of hearing problems, and selecting the group to which the test person's results are most similar.

14. A method according to claim 1 in which the person being tested responds to the presentation of target sounds by selecting with a computer mouse, or other pointing device, or touching on a touch screen, visual images that represent the correct response to the target sound presented, or by responding verbally, with such verbal response being recognized by voice recognition software within the test device.

15. A method according claim 3 in which the results for an individual test are derived by comparing the results of the test person to the results of a reference person who is believed to have normal hearing ability and who undergoes a test using the same computing device and headphones.

16. A method according to claim 4 in which the stimuli are made equally difficult by analyzing, across test participants, for each test item, the proportion of items correct as a function of its presentation level relative to the average presentation level for each participant, and then adjusting the level of each item so that the percentage correct for all the items have approximately the same dependence on presentation level relative to the average presentation level of all items.

17. A method according to claim 1 wherein the stimuli used to create the tests are an integral part of the game or games played during the test.

18. A method according to claim 17 wherein the game involves a story with characters or narrative or both, designed to maintain the interest, attention and concentration of the test person for the duration of the tests.

19. A method according to claim 1 in which the results of the test or tests are available to be displayed to the test person immediately on completion of the test.

20. A system for determining the hearing status of a person including:
means for administering more than one type of a hearing test in the context of a game or games.

21. (canceled)

22. (canceled)
